(19) United States
(12) Patent Application Publication
(10) Pub. No.: US 2009/0054818 A1
(43) Pub. Date: Feb. 26, 2009

(54) SPINAL ORTHOSES

(75) Inventors: Kenton R. Kaufman, Rochester, MN (US); Michael J. Yaszemski, Rochester, MN (US)

Correspondence Address: QUARLES & BRADY LLP, 411 E. WISCONSIN AVENUE, SUITE 2040, MILWAUKEE, WI, 53202-4497, US

(73) Assignee: MAYO FOUNDATION FOR MEDICAL EDUCATION AND RESEARCH, Rochester, MN (US)

(21) Appl. No.: 11/628,996

(22) PCT Filed: Jun. 9, 2005
(86) PCT No.: PCT/US2005/020401

§ 371 Date: Oct. 2, 2008

Related U.S. Application Data
(60) Provisional application No. 60/578,459, filed on Jun. 9, 2004.

(51) Int. Cl. A61F 5/00

(52) U.S. Cl. 602/19; 602/13; 602/16

(57) ABSTRACT

A thoracolumbosacral and a lumbosacral orthosis with sagittal-coronal control are disclosed. The orthoses have two rigid anterior and posterior plastic shells. The anterior shell extends from the pelvis to the sternum. The posterior shell extends from the pelvis and terminates just inferior to the scapular spine. An interior surface of the anterior shell has pressure pads that apply pressure to locations on the patient's anterior torso and the interior surface of the posterior shell has pressure pads that apply pressure to locations on the patient's posterior torso when the anterior shell and the posterior shell are secured to the patient's torso. The pressure pads may be inflatable air bladders.

### CROSS-REFERENCES TO RELATED APPLICATIONS

This application claims priority from U.S. Provisional Patent Application No. 60/578,459 filed Jun. 9, 2004.

### STATEMENT REGARDING FEDERALLY SPONSORED RESEARCH

Not Applicable.

### BACKGROUND OF THE INVENTION

1. Field of the Invention

This invention relates to spinal orthoses and more particularly to a thoracolumbosacral orthosis, a lumbosacral orthosis, a cervical-thoracic-lumbar-sacral orthosis, and a cervical orthosis.

2. Description of the Related Art

Spine bracing is used in a variety of acute and chronic conditions. It has been estimated that 1,688,000 people in United States wear a back brace. Typically, two types of back brace are used: (1) custom and (2) off-the-shelf, which are generally plastic or canvas. A custom back brace costs about $1,500, while an off-the-shelf back brace costs about $400. Virtually every spine surgeon in the United States uses back braces for the operative and deformity diagnoses, and it is estimated that many non-orthopedists (i.e., family practitioners, general practitioners, and internists who see people with back pain) use them.

Conditions treated with back braces include: low back pain (etiology unknown); thoracolumbar fracture; adolescent idiopathic scoliosis; adult (degenerative) scoliosis; spondylolysis, disc herniation; spondylolisthesis; and post-surgical immobilization (e.g., fracture, fusion for degenerative conditions, fusion for deformity). The efficacy of bracing of the lumbar spine is thought to be due to a complex interaction of several factors including but not limited to motion reduction, increased intra-abdominal pressure, and pressure feedback on musculature.

The three major reasons for prescribing spinal orthoses are: immobilization following spinal surgery, correction of spinal deformities, and alleviation of low back pain. As noted above, many types of spinal orthoses exist, ranging from simple, inexpensive cloth lumbosacral supports to expensive, custom-fitted thoracolumbosacral orthoses (TLSOs) with thigh extenders. Some example thoracolumbosacral orthoses are found in U.S. Pat. Nos. 5,718,670 and 5,362,304.

While the ability of orthoses to restrict individual intervertebral motions has been questioned (see Benzel et al., “Post-operative stabilization of a post-traumatic thoracic and lumbar spine: Interview of concepts and orthotic techniques.”, Journal of Spinal Disorders 2:47-51, 1989; and Axelsson et al., “Effect of lumbar orthosis on intervertebral mobility. A roentgen stereophotogrammetric analysis.”, Spine 17:678-681, 1992), recent research using video fluoroscopy to evaluate intervertebral motion has confirmed the stabilizing effect of a rigid custom-fitted TLSO (see Vander Kooi et al., “Lumbar Spine Stabilization with a Thoracolumbosacral Orthosis: Evaluation with Video Fluoroscopy.” Spine 29(1):100-104, 2004). In contrast, it has been reported that a lumbosacral corset is unable to immobilize the L3-S1 levels (see Miller et al., “Lower spine mobility in external immobilization in the normal and pathologic condition.”, Orthopedic Review 21(6): 753-757, 1992). Although custom-fitted TLSOs have been shown to be superior, they can be quite expensive.

Ideally, a back brace must meet several design criteria. A back brace should accommodate varying anthropometrics such as anterior/posterior (A/P) & medial/lateral (M/L) dimensions and an obese abdomen. A back brace should accommodate gender differences including breasts and hips. A back brace should have the ability to resist flexion moments, to add a thigh extender, and to accommodate spinal deformity. The ability to use the back brace as an adolescent scoliosis brace is also preferred.

Thus, there is a need for a thoracolumbosacral orthosis, a lumbosacral orthosis, a cervical-thoracic-lumbar-sacral orthosis, and a cervical collar orthosis that meet these design criteria and provide the stability of a custom fitted orthosis while also offering the cost effectiveness of an off-the-shelf brace.

### SUMMARY OF THE INVENTION

In a first aspect, the invention is directed to a thoracolumbosacral orthosis with sagittal-coronal control. The thoracolumbosacral orthosis is a modular segmented spinal system and has two rigid plastic shells. The anterior shelf extends from the pelvis to the sternum. The posterior shell extends from the pelvis (proximal sacrum) and terminates just inferior to the scapular spine. The thoracolumbosacral orthosis restricts gross trunk motion in the sagittal and coronal planes. Lateral strength is provided by overlapping plastic and stabilizing closures including straps and closures. The thoracolumbosacral orthosis is prefabricated and includes fitting and adjustment systems.

The thoracolumbosacral orthosis uses rigid anterior and posterior shells lined with inflatable air bladders that provide support and increased stability. The rigid shells can be manufactured in various sizes to accommodate the range of patient anthropometrics. The inflatable air bladders conform to the patient's anatomical topography and provide the stabilization required. The thoracolumbosacral orthosis of the invention is superior to the current methods because the rigid shells can be mass produced at a low expense while the inflatable air bladders provide the customizing effect which will yield the required stability.

In one example form, the thoracolumbosacral orthosis includes (i) an anterior shell dimensioned for covering an anterior portion of a patient's torso such that the anterior shell extends from approximately the patient's pelvis to approximately the patient's sternum, (ii) a posterior shell dimensioned for covering a posterior portion of the patient's torso such that the posterior shell extends from approximately the patient's pelvis and terminates inferior to approximately the patient's scapular spine, and (iii) a fastening system for securing the anterior shell in covering relationship to the anterior portion of the patient's torso and for securing the posterior shell in covering relationship to the posterior portion of the patient's torso.

This example thoracolumbosacral orthosis includes a pair of axillary anterior shell pressure pads secured to opposed upper lateral sections of the interior surface of the anterior shell, a sternal anterior shell pressure pad secured to an upper intermediate section of the interior surface of the anterior shell, a pair of rib anterior shell pressure pads secured to opposed intermediate lateral sections of the interior surface of the anterior shell, a pair of anterior superior iliac spine anterior shell pressure pads secured to opposed lower second lateral sections of the interior surface of the anterior shell, a pair of axillary posterior shell pressure pads secured to opposed upper lateral sections of the interior surface of the posterior shell, a scapular posterior shell pressure pad secured to an upper intermediate section of the interior surface of the posterior shell, a pair of rib posterior shell pressure pads secured to opposed intermediate lateral sections of the interior surface of the posterior shell, a lumbar posterior shell pressure pad secured to a lower intermediate section of the interior surface of the posterior shell, and a pair of spaced apart paraspinal posterior shell pressure pads secured to a central intermediate section of the interior surface of the posterior shell below the scapular posterior shell pressure pad. The pressure pads are preferably removable inflatable air bladders such that one or any number more than one of the pressure pads may be used to apply pressure to a location on the patient's anterior torso and to apply pressure to a location on the patient's posterior torso when the anterior shell and the posterior shell are secured to the patient's torso.

In another example form, the thoracolumbosacral orthosis includes an anterior thigh support, a posterior thigh support secured to and extending downward from the posterior shell, and a fastening system for securing the anterior thigh support in covering relationship to an anterior portion of the patient's thigh and for securing the posterior thigh support in covering relationship to a posterior portion of the patient's thigh. The posterior thigh support may be secured to the posterior shell with a hinge. In one form, a pair of laterally spaced anterior thigh support pressure pads extend away from an interior surface of the anterior thigh support, and a pair of laterally spaced posterior thigh support pressure pads extend away from an interior surface of the posterior thigh support.

In a second aspect, the present invention provides a lumbosacral orthosis that uses rigid anterior and posterior shells lined with inflatable air bladders that provide support and increased stability. The rigid shells can be manufactured in various sizes to accommodate the range of patient anthropometrics. The inflatable air bladders conform to the patient's anatomical topography and provide the stabilization required. The lumbosacral orthosis of the invention is superior to the current methods because the rigid shells can be mass produced at a low expense while the inflatable air bladders provide the customizing effect which will yield the required stability.

In one example form, the lumbosacral orthosis includes (i) an anterior shell dimensioned for covering an anterior portion of a patient's torso such that the anterior shell extends from approximately the patient's pelvis to approximately a lower portion of the sternum, i.e. xiphoid process, (ii) a posterior shell dimensioned for covering a posterior portion of the patient's torso such that the posterior shell extends from approximately the patient's pelvis and terminates at the inferior portion of the scapula (at about the same level as the patient's xiphoid process), and (iii) a fastening system for securing the anterior shell in covering relationship to the anterior portion of the patient's torso and for securing the posterior shell in covering relationship to the posterior portion of the patient's torso.

This example lumbosacral orthosis includes a pair of rib anterior shell pressure pads secured to opposed intermediate lateral sections of the interior surface of the anterior shell, a pair of anterior superior iliac spine anterior shell pressure pads secured to opposed lower second lateral sections of the interior surface of the anterior shell, a pair of rib posterior shell pressure pads secured to opposed intermediate lateral sections of the interior surface of the posterior shell, a lumbar posterior shell pressure pad secured to a lower intermediate section of the interior surface of the posterior shell, and a pair of spaced apart paraspinal posterior shell pressure pads secured to a central intermediate section of the interior surface of the posterior shell. The pressure pads are preferably removable inflatable air bladders such that one or any number more than one of the pressure pads may be used to apply pressure to a location on the patient's anterior torso and to apply pressure to a location on the patient's posterior torso when the anterior shell and the posterior shell are secured to the patient's torso.

In a third aspect, the invention provides a cervical-thoracic-lumbar-sacral orthosis. This orthosis includes the anterior shell, the posterior shell, and any number of the pressure pads of the thoracolumbosacral orthosis of the first aspect of the invention. The cervical-thoracic-lumbar-sacral orthosis further includes a mandibular support secured to and extending upward from the anterior shell, and at least one mandibular support pressure pad extending away from an interior surface of the mandibular support. The cervical-thoracic-lumbar-sacral orthosis further includes an occipital support secured to and extending upward from the posterior shell, and at least one occipital support pressure pad extending away from an interior surface of the occipital support. In one example form, the cervical-thoracic-lumbar-sacral orthosis includes a pair of laterally spaced apart mandibular support pressure pads extending away from the interior surface of the mandibular support, and a pair of laterally spaced apart occipital support pressure pads extending away from the interior surface of the occipital support; The pressure pads are preferably removable inflatable air bladders such that one or any number more than one of the pressure pads may be used to apply pressure to a location on the patient's mandible and to apply pressure to a location on the patient's occipital region when the anterior shell and the posterior shell are secured to the patient.

In a fourth aspect, the invention provides a cervical collar orthosis including (i) an anterior shell having a mandibular support region dimensioned for contacting a patient's mandible and having a clavicular support region dimensioned for contacting the patient's collar bones, (ii) a posterior shell having an occipital support region dimensioned for contacting the patient's occipital bones and having a lower posterior support region dimensioned for contacting the patient's scapular spine or the posterior portion of the patient's neck, and (iii) a fastening system for securing the anterior shell in covering relationship to an anterior portion of the patient's neck region and for securing the posterior shell in covering relationship to a posterior portion of the patient's neck region.

In one example form, the cervical collar orthosis has a pair of laterally spaced mandibular support pressure pads extending away from the interior surface of the anterior shell at the mandibular support region of the anterior shell, a pair of laterally spaced clavicular support pressure pads extending away from the interior surface of the anterior shell at the clavicular support region of the anterior shell, a pair of laterally spaced occipital support pressure pads extending away from the interior surface of the posterior shell at the occipital support region of the posterior shell, and a pair of laterally spaced lower posterior support pressure pads extending away from the interior surface of the posterior shell at the lower posterior support region of the posterior shell. The pressure pads are preferably removable inflatable air bladders such that one or any number more than one of the pressure pads may be used to apply pressure to a location on the patient's mandible, collar bones, occipital region, posterior neck, and/or scapular region when the anterior shell and the posterior shell are secured to the patient.

These and other features, aspects, and advantages of the present invention will become better understood upon consideration of the following detailed description, drawings, and appended claims.

### BRIEF DESCRIPTION OF THE DRAWINGS

FIG. 1 is a front perspective view showing a thoracolumbosacral orthosis according to the invention applied to the torso of a patient.

FIG. 2 is a rear perspective view showing the thoracolumbosacral orthosis of FIG. 1 applied to the torso of a patient.

FIG. 3 is a left side view showing the thoracolumbosacral orthosis of FIG. 1 applied to the torso of a patient.

FIG. 4 is a view taken along line 4-4 of FIG. 3 showing the interior surface of the anterior shell of the thoracolumbosacral orthosis.

FIG. 5 is a view taken along line 5-5 of FIG. 3 showing the interior surface of the posterior shell of the thoracolumbosacral orthosis.

FIG. 6 is a front perspective view showing a cervical-thoracic-lumbar-sacral orthosis according to the invention applied to the torso of a patient.

FIG. 7 is a left side view showing the cervical-thoracic-lumbar-sacral orthosis of FIG. 6 applied to the torso of a patient.

FIG. 8 is a view taken along line 8-8 of FIG. 7 showing the interior surface of the mandibular support shell of the cervical-thoracic-lumbar-sacral orthosis.

FIG. 9 is a view taken along line 9-9 of FIG. 7 showing the interior surface of the occipital support shell of the cervical-thoracic-lumbar-sacral orthosis.

FIG. 10 is a front perspective view showing a thoracolumbosacral orthosis with a thigh extender according to the invention applied to the torso of a patient.

FIG. 11 is a rear perspective view showing a thoracolumbosacral orthosis with a thigh extender of FIG. 10 applied to the torso of a patient.

FIG. 12 is a left side view showing a thoracolumbosacral orthosis with a thigh extender of FIG. 10 applied to the torso of a patient.

FIG. 13 is a view taken along line 13-13 of FIG. 12 showing the interior surface of the anterior thigh extender shell of the thoracolumbosacral orthosis with a thigh extender.

FIG. 14 is a view taken along line 14-14 of FIG. 12 showing the interior surface of the posterior thigh extender shell of the thoracolumbosacral orthosis with a thigh extender.

FIG. 15 is a front perspective view showing a cervical collar orthosis according to the invention.

FIG. 16 is an elevational view showing the interior surface of the anterior shell of the cervical collar orthosis of FIG. 15.

FIG. 17 is an elevational view showing the interior surface of the posterior shell of the cervical collar orthosis of FIG. 15.

FIG. 18 is a front perspective view showing a lumbosacral orthosis according to the invention applied to the torso of a patient.

FIG. 19 is a rear perspective view showing the lumbosacral orthosis of FIG. 18 applied to the torso of a patient.

FIG. 20 is a left side view showing the lumbosacral orthosis of FIG. 18 applied to the torso of a patient.

FIG. 21 is a view taken along line 21-21 of FIG. 20 showing the interior surface of the anterior shell of the lumbosacral orthosis.

FIG. 22 is a view taken along line 22-22 of FIG. 20 showing the interior surface of the posterior shell of the lumbosacral orthosis.

Like reference numerals will be used to refer to like parts from Figure to Figure in the following description of the drawings.

### DETAILED DESCRIPTION OF THE INVENTION

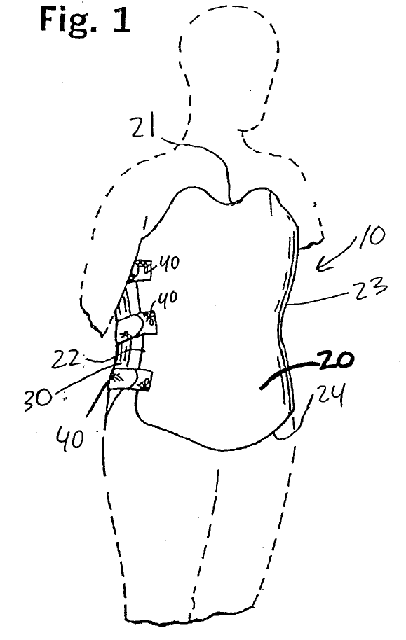

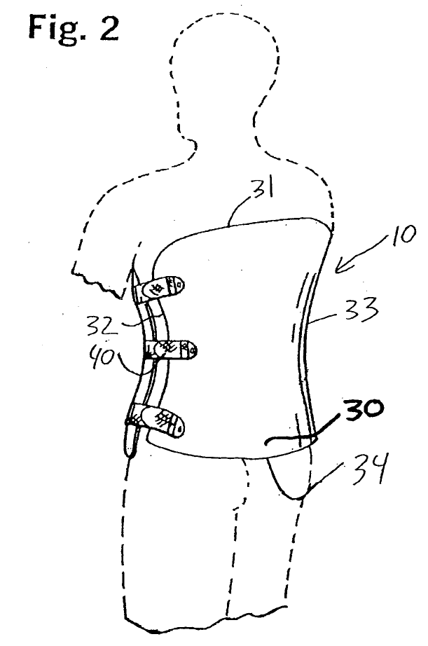

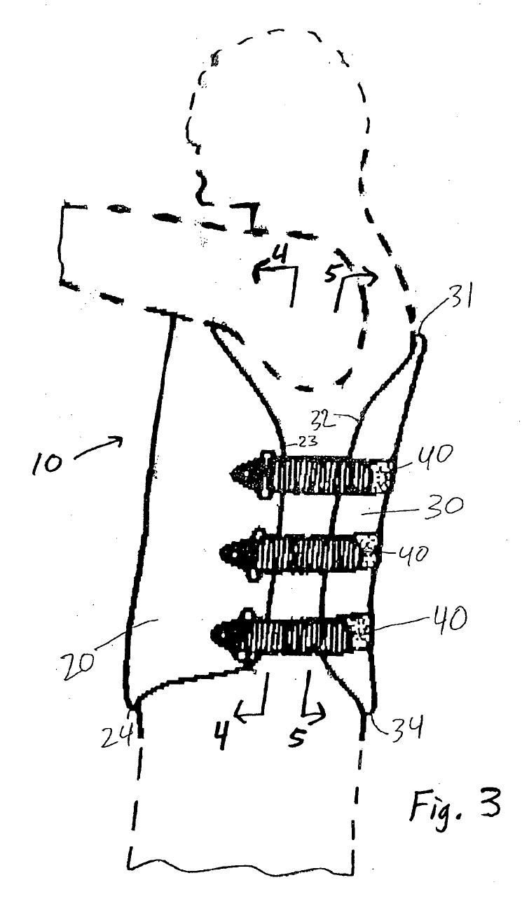

Referring first to FIGS. 1-3, there is shown one example embodiment of a thoracolumbosacral orthosis according to the invention for overlying the torso of a patient. The thoracolumbosacral orthosis is generally indicated at 10, and includes an anterior shell 20 and a posterior shell 30 for overlying the anterior and posterior trunk portions of the torso of the patient. The anterior shell 20 and the posterior shell 30 are each dimensioned to the shape of the anterior portion and the posterior portion of the torso, respectively, such that the anterior shell 20 and the posterior shell 30 generally follow the three-dimensional contour of the torso in relationship to an interior surface 25 of the anterior shell 20 and an interior surface 35 of the posterior shell 30. The anterior shell 20 has a different convexity to accommodate differing abdomen and breast sizes. Optionally, the anterior shell 20 and the posterior shell 30 may comprise more than one piece. Also, the anterior shell 20 and the posterior shell 30 may be formed from a right shell and left shell with fasteners in the front and back. Specifically, the anterior portion of a left shell could form the left side of the anterior shell and the anterior portion of a right shell could form the right side of the anterior shell. Likewise, the posterior portion of a left shell could form the left side of the posterior shell and the posterior portion of a right shell could form the right side of the posterior shell.

The anterior shell 20 has a top edge 21, a first side edge 22, a second side edge 23, and a bottom edge 24. The posterior shell 30 has a top edge 31, a first side edge 32, a second side edge 33, and a bottom edge 34. Any of the top edge 21, the first side edge 22, the second side edge 23, and the bottom edge 24 of the anterior shell 20 may flair outwardly from the torso to change the stiffness and/or reduce chaffing on the torso. Likewise, any of the top edge 31, the first side edge 32, the second side edge 33, and the bottom edge 34 of the posterior shell 30 may flair outwardly from the torso to change the stiffness and/or reduce chaffing on the torso.

The anterior shell 20 and the posterior shell 30 are formed of a rigid material, and the stiffness of the anterior shell 20 and the posterior shell 30 may be changed by changing the type and/or thickness of materials. Preferably, each shell is formed of a plastic material such as a molded polyethylene or polypropylene. Preferably, at least five different sizes of the shells may be provided, for example, extra small, small, medium, large and extra large, or optionally, further sizes in-between may be provided, to accommodate the torsos of individuals of different sizes including variations in height and girth. Thus, multiple sizes for the anterior shell 20 and the posterior shell 30 are provided, and the sizes for the anterior shell 20 and the posterior shell 30 are interchangeable.

The anterior shell 20 and the posterior shell 30 are secured on opposite sides of the patient's torso by a fastening system 40 interconnecting the anterior shell 20 and the posterior shell 30. While many different types of fastening systems may be employed to secure the shells about the torso, Velcro™-type straps are preferred. One end of each strap is secured to one of the shells. The straps extend to mating portions of the Velcro™ fastener secured on the opposite shell. Alternative fastening systems include strap/buckle combinations and elastic materials.

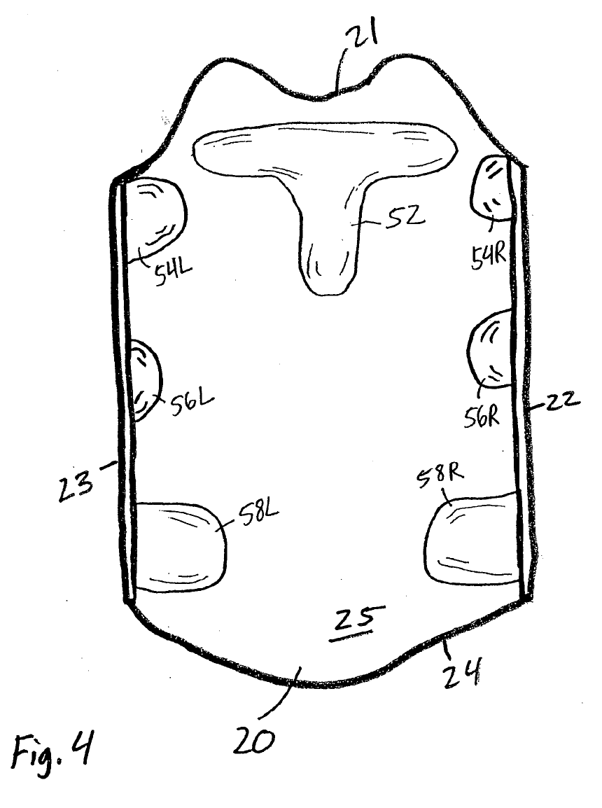

Turning now to FIG. 4, there is shown a view of the interior surface 25 of the anterior shell 20. Attached to the interior surface 25 of the anterior shell 20 are a number of anterior pressure pads. A T-shaped sternal-clavicular pad 52 is attached to the interior surface 25 of the anterior shell 20 at a top portion of the interior surface 25. Alternatively, the sternal-clavicular pad may be replaced with one of an oblong vertical sternal pad or an oblong horizontal clavicular pad. A pair of axillary pads 54L and 54R are attached to the interior surface 25 of the anterior shell 20 at opposite sides of the top portion of the interior surface 25. A pair of rib pads 56L and 56R are attached to the interior surface 25 of the anterior shell 20 at opposite sides of the middle portion of the interior surface 25. A pair of anterior superior iliac spine (ASIS) pads 58L and 58R are attached to the interior surface 25 of the anterior shell 20 at opposite sides of the bottom portion of the interior surface 25. Each of the anterior pressure pads, which are the sternal-clavicular pad 52, the pair of axillary pads 54L,54R, the pair of rib pads 56L,56R, and the pair of anterior superior iliac spine (ASIS) pads 58L,58R in the embodiment of the thoracolumbosacral orthosis 10 shown, extend away from the interior surface 25 of the anterior shell 20.

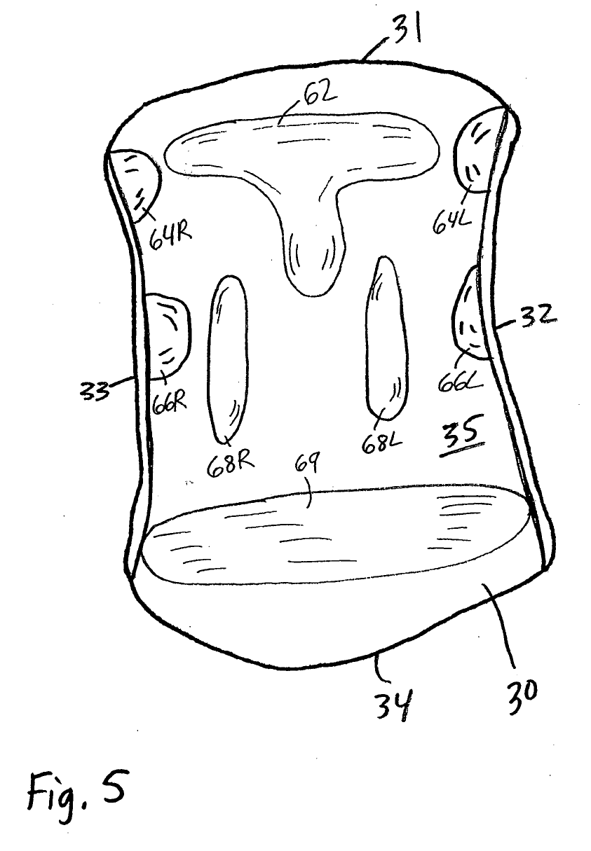

Turning now to FIG. 5, there is shown a view of the interior surface 35 of the posterior shell 30. Attached to the interior surface 35 of the posterior shell 30 are a number of posterior pressure pads. A T-shaped transscapular-interscapular pad 62 is attached to the interior surface 35 of the posterior shell 30 at a top portion of the interior surface 35. Alternatively, the transscapular-interscapular pad may be replaced with one of an oblong horizontal transscapular pad or an oblong vertical interscapular pad. A pair of axillary pads 64L and 64R are attached to the interior surface 35 of the posterior shell 30 at opposite sides of the top portion of the interior surface 35. A pair of rib pads 66L and 66R are attached to the interior surface 35 of the posterior shell 30 at opposite sides of the middle portion of the interior surface 35. A pair of paraspinal pads 68L and 68R are attached to the interior surface 35 of the posterior shell 30 at opposed positions at the interior of the middle portion of the interior surface 35. A lumbar pad 69 is attached to the interior surface 35 of the posterior shell 30 at the bottom portion of the interior surface 35. Each of the posterior pressure pads, which are the transscapular-interscapular pad 62, the pair of axillary pads 64L,64R, the pair of rib pads 66L,66R, the pair of paraspinal pads 68L, 68R, and the lumbar pad 69 in the embodiment of the thoracolumbosacral orthosis 10 shown, extend away from the interior surface 35 of the posterior shell 30.

In the preferred embodiment of the thoracolumbosacral orthosis 10, the anterior pressure pads each comprise an inflatable air bladder that is attached to the interior surface 25 of the anterior shell 20. Likewise, the posterior pressure pads each comprise an inflatable air bladder that is attached to the interior surface 35 of the posterior shell 30. While the anterior pressure pads and the posterior pressure pads each preferably comprise an inflatable air bladder, the invention is not limited to this type of anterior pressure pad and posterior pressure pad. While in the preferred embodiment, the anterior pressure pads and the posterior pressure pads are inflatable air bladders, the anterior pressure pads and the posterior pressure pads may also comprise liquid-filled sacs, gel-filled sacs or foam-type (e.g., polyurethane) pads.

Various means for attaching the anterior pressure pads to the interior surface 25 of the anterior shell 20 and for attaching the posterior pressure pads to the interior surface 35 of the posterior shell 30 can be provided. In one example, the means for attaching the anterior pressure pads to the interior surface 25 of the anterior shell 20 comprise (i) male Velcro™-type straps that are attached to or in-molded into the interior surface 25 of the anterior shell 20 at the desired locations for the anterior pressure pads and (ii) female Velcro™-type straps that are attached to each anterior pressure pad such that the anterior pressure pads may be attached to the interior surface 25 of the anterior shell 20 at the desired location by way of the typical Velcro™-type fastening action. In this example configuration, each anterior pressure pad can be readily attached or removed from the interior surface 25 of the anterior shell 20 depending on the individual patient's needs. Therefore, each of the anterior pressure pads shown in FIG. 4 may not be used in a treatment program. It can be appreciated that the same means for attaching the anterior pressure pads to the interior surface 25 of the anterior shell 20 would be advantageous for attaching the posterior pressure pads to the interior surface 35 of the posterior shell 30. Thus, each of the posterior pressure pads shown in FIG. 5 may not be used in a treatment program.

The thoracolumbosacral orthosis 10 including inflatable air bladders for the anterior pressure pads and the posterior pressure pads may be custom fitted to a patient's torso as follows. First, a size for the anterior shell 20 and a size for the posterior shell 30 are separately selected based on the patient's anatomy.

Next, a fixation plan is determined. When the thoracolumbosacral orthosis 10 is to be used for the treatment of back pain or for post-surgical treatment, a three-point fixation in the sagittal plane including (i) ASIS bilateral fixation, (ii) sternal fixation, and (iii) transscapular-interscapular and/or lumbar fixation is typically selected. This type of three-point fixation can be implemented in the thoracolumbosacral orthosis 10 by: (1) inflating the air bladders comprising the pair of anterior superior iliac spine (ASIS) pads 58L and 58R on the interior surface 25 of the anterior shell 20 such that the pair of anterior superior iliac spine (ASIS) pads 58L and 58R extends away from the interior surface 25 to create pressure pads that extends inwardly toward the patient's torso when the thoracolumbosacral orthosis 10 is placed on the patient; (2) inflating the air bladder comprising the sternal-clavicular pad 52 on the interior surface 25 of the anterior shell 20 such that the sternal-clavicular pad 52 extends away from the interior surface 25 to create a pressure pad that extends inwardly toward the patient's torso; (3) inflatirig the air bladders comprising the transscapular-interscapular pad 62 and/or the lumbar pad 69 on the interior surface 35 of the posterior shell 30 such that the transscapular-interscapular pad 62 and/or the lumbar pad 69 extends away from the interior surface 35 to create a pressure pad that extends inwardly toward the patient's torso. Of course, any other anterior or posterior pressure pads may be inflated as needed under the patient treatment program. For example, the paraspinal pads 68L and 68R may be inflated. The anterior shell 20 and the posterior shell 30 are then secured to the patient's torso as shown in FIGS. 1-3 using the fastening system 40. Alternatively, the anterior shell 20 and the posterior shell 30 may be secured to the patient's torso first and then the air bladders for the anterior pressure pads and the posterior pressure pads may be inflated thereafter for custom fitting to a patient's torso.

When the thoracolumbosacral orthosis 10 is to be used for the treatment of scoliosis, a three-point fixation in the coronal plane including (i) unilateral iliac crest fixation, (ii) axillary fixation, and (iii) contralateral apex fixation is typically selected. This type of three-point fixation can be implemented in the thoracolumbosacral orthosis 10 by: (1) inflating one of the air bladders comprising the pair of anterior superior iliac spine (ASIS) pads (e.g., 58L) on the interior surface 25 of the anterior shell 20 such that the anterior superior iliac spine (ASIS) pad (e.g., 58L) extends away from the interior surface 25 to a create pressure pad that extend inwardly toward the patient's torso when the thoracolumbosacral orthosis 10 is placed on the patient; (2) inflating one of the air bladders comprising the pair of axillary pads (e.g. 54L) on the interior surface 25 of the anterior shell 20 such that the axillary pad (e.g., 54L) extends away from the interior surface 25; (3) inflating one of the air bladders comprising the pair of axillary pads (e.g., 64L) on the interior surface 35 of the posterior shell 30 such that the axillary pad (e.g., 64L) extends away from the interior surface 35 to a create a pressure pad; (4) inflating one of the air bladders comprising the pair of rib pads (e.g. 56R) on the interior surface 25 of the anterior shell 20 such that the rib pad (e.g., 56R) extends away from the interior surface 25; (4) inflating one of the air bladders comprising the pair of rib pads (e.g. 66R) on the interior surface 35 of the posterior shell 30 such that the rib pad (e.g., 66R) extends away from the interior surface 25; and (5) inflating the air bladder comprising the lumber pad 69 on the interior surface 35 of the posterior shell 30 such that the lumbar pad 69 extends away from the interior surface 35 to create a pressure pad. Of course, any other anterior or posterior pressure pads may be inflated as needed under the patient treatment program.

The anterior shell 20 and the posterior shell 30 are then secured to the patient's torso as shown in FIGS. 1-3 using the fastening system 40. It can be seen that: (i) the inflated anterior superior iliac spine (ASIS) pad 58L and the lumbar pad 69 create unilateral (left side) iliac crest fixation, (ii) the axillary pads 54L and 64L create axillary (left side) fixation, and (iii) the rib pads 56R and 66R create contralateral apex (right side) fixation. Of course, the sides of the unilateral iliac crest fixation, the axillary fixation, and the contralateral apex fixation can be reversed by appropriate inflation of the opposite air bladders. Also, an alternative lumbar pad that is limited to one side of the posterior shell 30 may be used. Alternatively, the anterior shell 20 and the posterior shell 30 may be secured to the patient's torso first and then the air bladders for the anterior pressure pads and the posterior pressure pads may be inflated thereafter for custom fitting to a patient's torso.

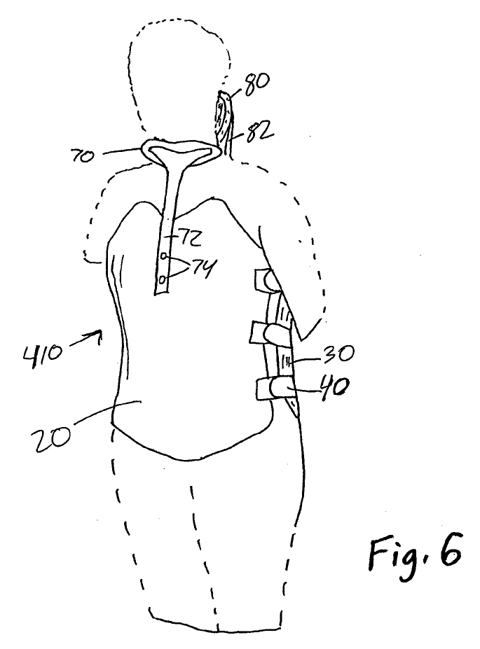

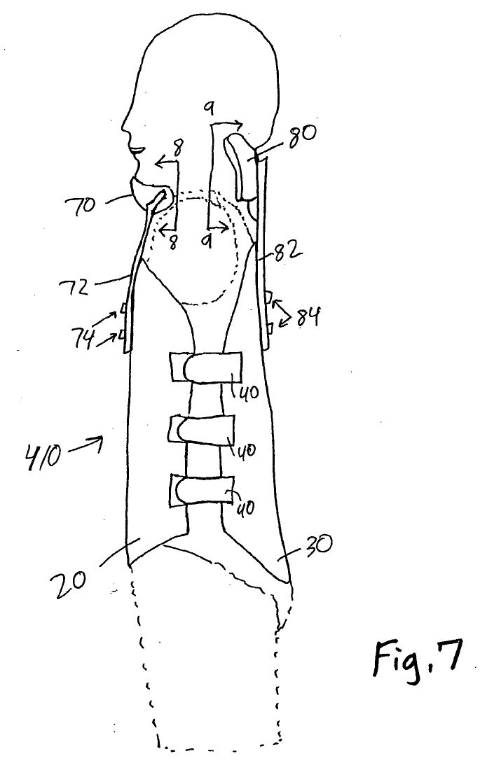

Referring now to FIGS. 6-9, there is shown a cervical-thoracic-lumbar-sacral orthosis 410 according to the invention applied to the torso of a patient. The cervical-thoracic-lumbar-sacral orthosis 410 includes the anterior shell 20, the posterior shell 30, the fastening system 40 interconnecting the anterior shell 20 and the posterior shell 30, the anterior pressure pads, and the posterior pressure pads as described above with reference to FIGS. 1-5.

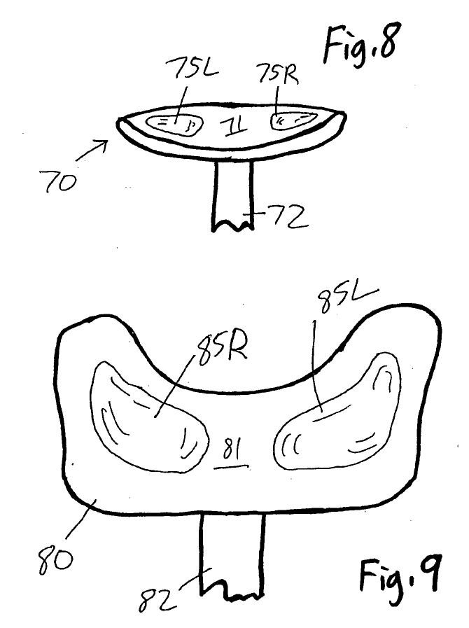

The cervical-thoracic-lumbar-sacral orthosis 410 further includes an arcuate rigid mandibular support 70 secured to the anterior shell 20 by way of a support arm 72 and fasteners 74 such as screws. The mandibular support 70 typically is formed from a plastic material such as a molded polyethylene or polypropylene, and the support arm 72 and fasteners 74 are typically metallic. The mandibular support 70 applies pressure to the mandible bones by way of a pair of laterally spaced apart mandibular support pressure pads 75L and 75R (shown in FIG. 8) that extend away from an interior surface 71 of the mandibular support 70.

The cervical-thoracic-lumbar-sacral orthosis 410 further includes a rigid occipital support 80 secured to the posterior shell 30 by way of a support arm 82 and fasteners 84 such as screws. The occipital support 80 typically is formed from a plastic material such as a molded polyethylene or polypropylene, and the support arm 82 and fasteners 84 are typically metallic. The occipital support 80 applies pressure to the occipital bones by way of a pair of laterally spaced apart occipital support pressure pads 85L and 85R (shown in FIG. 9) that extend away from an interior surface 81 of the occipital support 80.

In the preferred embodiment of the cervical-thoracic-lumbar-sacral orthosis 410, the mandibular support pressure pads 75L and 75R each comprise an inflatable air bladder that is attached to the interior surface 71 of the mandibular support 70. Likewise, the occipital support pressure pads 85L and 85R each comprise an inflatable air bladder that is attached to the interior surface 81 of the occipital support 80. The mandibular support pressure pads 75L and 75R and the occipital support pressure pads 85L and 85R may also comprise liquid-filled sacs, gel-filled sacs or foam-type (e.g., polyurethane) pads.

Various means for attaching the mandibular support pressure pads 75L and 75R to the interior surface 71 of the mandibular support 70 can be provided. In one example, the means for attaching comprise (i) male Velcro™-type straps that are attached to or in-molded into the interior surface 71 of the mandibular support 70 at the desired locations for the mandibular support pressure pads 75L and 75R and (ii) female Velcro™-type straps that are attached to each of the mandibular support pressure pads 75L and 75R such that the mandibular support pressure pads 75L and 75R may be attached to the interior surface 71 of the mandibular support 70 at the desired location by way of the typical Velcro™-type fastening action. In this example configuration, each mandibular support pressure pad 75L and 75R can be readily attached or removed from the interior surface 71 of the mandibular support 70 depending on the individual patient's needs. It can be appreciated that the same means for attaching the mandibular support pressure pads 75L and 75R to the interior surface 71 of the mandibular support 70 would be advantageous for attaching the occipital support pressure pads 85L and 85R to the interior surface 81 of the occipital support 80. Thus, each of the occipital support pressure pads 85L and 85R can be readily attached or removed from the interior surface 81 of the occipital support 80 depending on the individual patient's needs.

The cervical-thoracic-lumbar-sacral orthosis 410 helps stabilize or support the pelvis, back, neck and head. It is used to support weakened or damaged areas of the spine and to stabilize and control unwanted curvatures of the spine (scoliosis). Patients who use prior art cervical-thoracic-lumbar-sacral devices sometimes get decubital ulcers at the base of their skull due to the prolonged pressure applied to the skin. The air bladder concept provides stability while reducing the pressures, and thus reduces complications associated with application of a cervical-thoracic-lumbar-sacral orthosis. The mandibular support pressure pads 75L and 75R and the occipital support pressure pads 85L and 85R are arranged bilaterally. The mandibular support pressure pads 75L and 75R and the occipital support pressure pads 85L and 85R apply pressure on the mandible and occipital bones. The mandibular support pressure pads 75L and 75R may include an option to be deflated while the subject is eating so that they can chew.

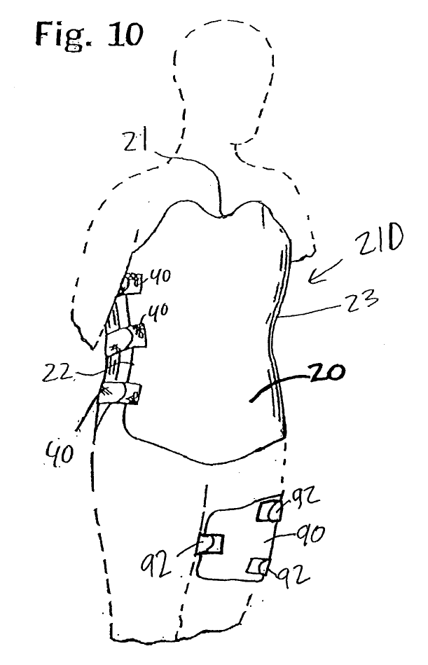

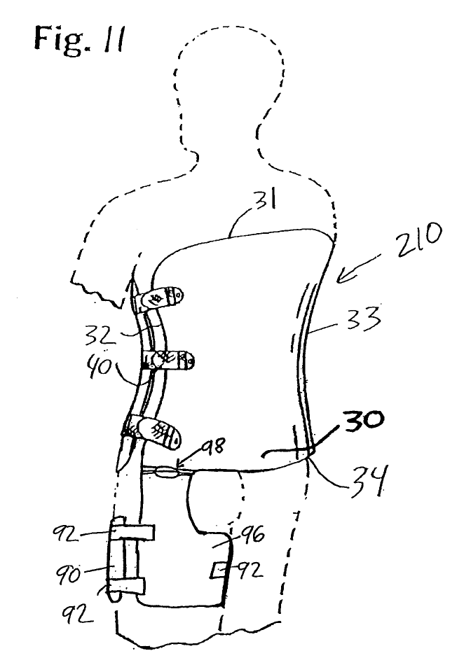

Turning now to FIGS. 10-14, there is shown a thoracolumbosacral orthosis 210 having a thigh extender which adds stability to the thoracolumbosacral orthosis 210. The thoracolumbosacral orthosis 210 includes an anterior thigh support 90, and a posterior thigh support 96 that is secured to and extends downward from the posterior shell 30. The posterior thigh support 96 is secured to the posterior shell 30 with a hinge 98. Preferably, the hinge 98 is positioned directly over the hip center to allow motion to occur. The hinge 98 is an optional feature of the thoracolumbosacral orthosis 210. The anterior thigh support 90 and the posterior thigh support 96 are interconnected around the patient's thigh by a fastening system 92. While many different types of fastening systems may be employed to secure the anterior thigh support 90 and the posterior thigh support 96 about the thigh, Velcro™-type straps are preferred. One end of each strap is secured to one of the anterior thigh support 90 and the posterior thigh support 96. The anterior thigh support 90 and the posterior thigh support 96 are typically formed from a plastic material such as a molded polyethylene or polypropylene. In another form, the anterior thigh support 90 is secured to the anterior shell 30 with a hinge, and the posterior thigh support 96 is not connected to the posterior shell 30.

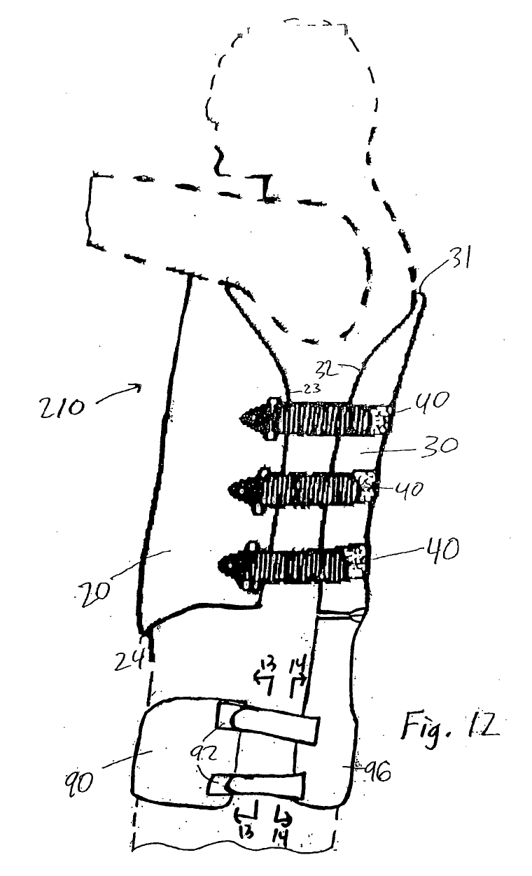

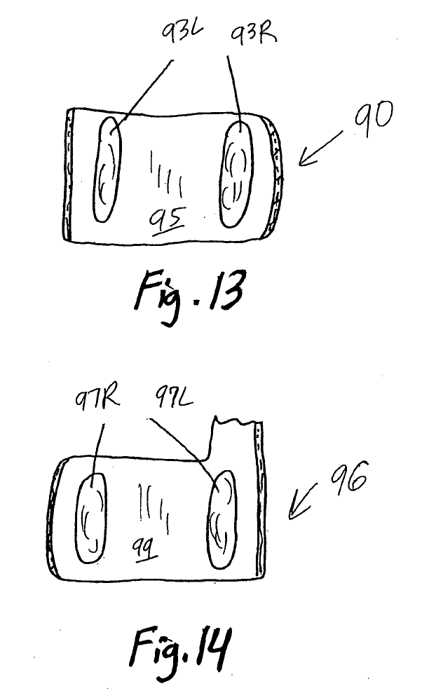

Looking at FIG. 13, the anterior thigh support 90 has a pair of laterally spaced apart anterior thigh support pressure pads 93L and 93R extending away from an interior surface 95 of the anterior thigh support 90. Likewise, in FIG. 14, the posterior thigh support 96 has a pair of laterally spaced apart posterior thigh support pressure pads 97L and 97R extending away from an interior surface 99 of the posterior thigh support 96. The anterior thigh support pressure pads 93L and 93R and the posterior thigh support pressure pads 97L and 97R may each comprise an inflatable air bladder, liquid-filled sacs, gel-filled sacs or foam-type (e.g., polyurethane) pads. The anterior thigh support pressure pads 93L and 93R and the posterior thigh support pressure pads 97L and 97R are optional in that the thigh already has enough soft tissue mass that many shells would readily conform to the thigh and give good fixation.

Various means for attaching the anterior thigh support pressure pads 93L and 93R to the interior surface 95 of the anterior thigh support 90 can be provided. In one example, the means for attaching comprise (i) male Velcro™-type straps that are attached to or in-molded into the interior surface 95 of the anterior thigh support 90 at the desired locations and (ii) female Velcro™-type straps that are attached to each of the anterior thigh support pressure pads 93L and 93R such that the anterior thigh support pressure pads 93L and 93R may be attached to the interior surface 95 of the anterior thigh support 90 at the desired location by way of the typical Velcro™-type fastening action. It can be appreciated that the same means for attaching the anterior thigh support pressure pads 93L and 93R to the interior surface 95 of the anterior thigh support 90 would be advantageous for attaching the posterior thigh support pressure pads 97L and 97R to the interior surface 99 of the posterior thigh support 96.

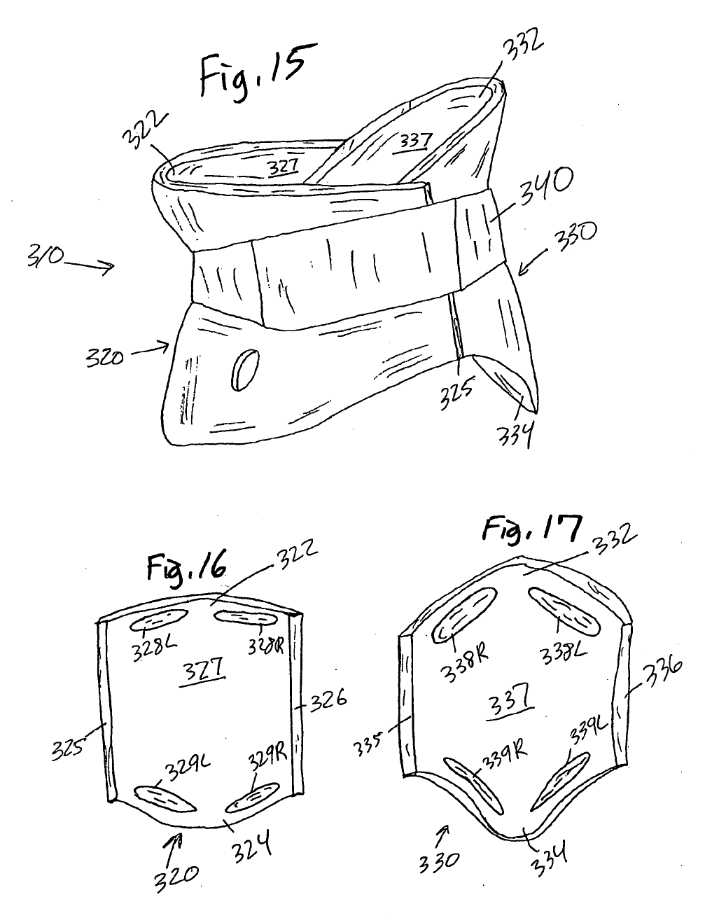

Referring now to FIGS. 15-17, there is shown a cervical collar orthosis 310 according to the Invention. The cervical collar orthosis 310 includes an anterior support shell 320, and a posterior support shell 330. The anterior support shell 320 and the posterior support shell 330 are interconnected around the patient's neck region by a fastening system 340. While many different types of fastening systems may be employed to secure the anterior support shell 320 and the posterior support shell 330 about the neck region, Velcro™-type straps are preferred. One end of each strap is secured to one of the anterior support shell 320 and the posterior support shell 330. The anterior support shell 320 and the posterior support shell 330 are typically formed from a plastic material such as a molded polyethylene or polypropylene or polyurethane. The cervical collar orthosis 310 includes inflatable air bladders (liquid-filled sacs, gel-filled sacs or foam-type (e.g., polyurethane) pads) positioned bilaterally on the mandible and occipital bone, and inflatable air bladders (liquid-filled sacs, gel-filled sacs or foam-type (e.g., polyurethane) pads) positioned bilaterally on the lower portion of the cervical collar orthosis 310 where the cervical collar orthosis 310 touches the base of the neck.

Looking at FIG. 16, the anterior support shell 320 of the cervical collar orthosis 310 is generally arcuate, and has a mandibular support region 322, a clavicular support region 324, and opposed sides 325, 326. The mandibular support region 322 applies pressure to the mandible bones of a patient by way of a pair of laterally spaced apart mandibular support pressure pads 328L and 328R attached to the interior surface 327 (which faces the body of the patient) of the anterior support shell 320 in the mandibular support region 322. The clavicular support region 324 applies pressure to the collar bones of a patient by way of a pair of laterally spaced apart clavicular support pressure pads 329L and 329R attached to the interior surface 327 of the anterior support shell 320 in the clavicular support region 324.

Looking at FIG. 17, the posterior support shell 330 of the cervical collar orthosis 310 is generally arcuate, and has an occipital support region 332, a lower posterior support region 334, and opposed sides 335, 336. The occipital support region 332 applies pressure to the occipital bones of a patient by way of a pair of laterally spaced apart occipital support pressure pads 338L and 338R attached to the interior surface 337 (which faces the body of the patient) of the posterior support shell 330 in the occipital support region 332. The lower posterior support region 334 applies pressure to the scapular spine and/or the neck of a patient by way of a pair of laterally spaced apart lower posterior support pressure pads 339L and 339R attached to the interior surface 327 of the posterior support shell 330 in the lower posterior support region 334.

Various means for attaching the mandibular support pressure pads 328L and 328R and the clavicular support pressure pads 329L and 329R to the interior surface 327 of the anterior support shell 320 can be provided. In one example, the means for attaching comprise (i) male Velcro™-type straps that are attached to or in-molded into the interior surface 327 of the anterior support shell 320 at the desired locations and (ii) female Velcro™-type straps that are attached to each of the mandibular support pressure pads 328L and 328R and the clavicular support pressure pads 329L and 329R such that the mandibular support pressure pads 328L and 328R and the clavicular support pressure pads 329L and 329R may be attached to the interior surface 327 of the anterior support shell 320 at the desired location by way of the typical Velcro™-type fastening action. It can be appreciated that the same means for attaching would be advantageous for attaching the occipital support pressure pads 338L and 338R and the lower posterior support pressure pads 339L and 339R to the interior surface 337 of the posterior support shell 330.

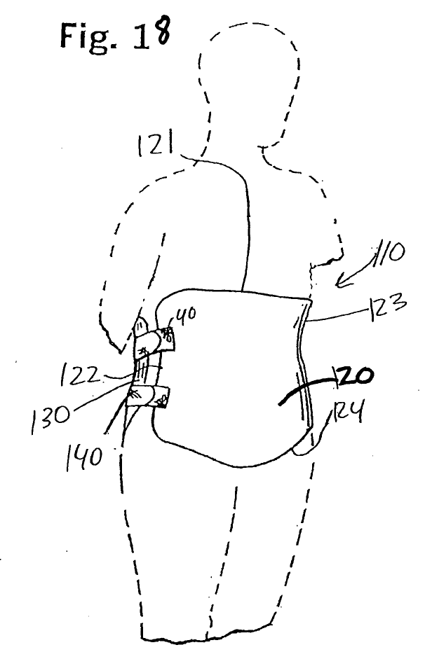

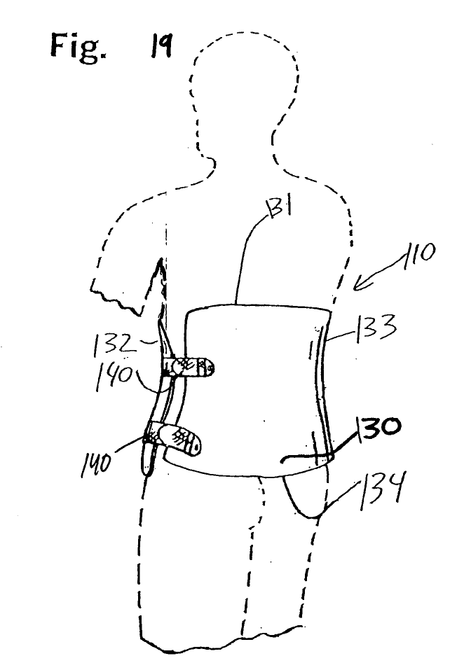

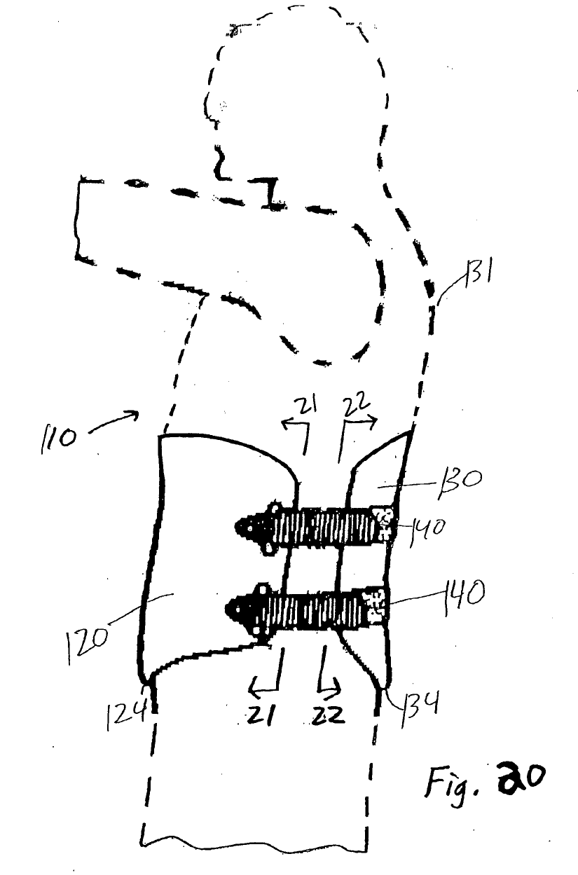

Turning to FIGS. 18-22, there is shown one example embodiment of a lumbosacral orthosis according to the invention for overlying the torso of a patient. The lumbosacral orthosis is generally indicated at 110, and includes an anterior shell 120 and a posterior shell 130 for overlying the anterior and posterior trunk portions of the torso of the patient. The anterior shell 120 and the posterior shell 130 are each dimensioned to the shape of the anterior portion and the posterior portion of the torso, respectively, such that the anterior shell 120 and the posterior shell 130 generally follow the three-dimensional contour of the torso in relationship to an interior surface 125 of the anterior shell 120 and an interior surface 135 of the posterior shell 130. The anterior shell 120 has a different convexity to accommodate differing abdomen sizes. Optionally, the anterior shell 120 and the posterior shell 130 may comprise more than one piece.

The anterior shell 120 has a top edge 121, a first side edge 122, a second side edge 123, and a bottom edge 124. The posterior shell 130 has a top edge 131, a first side edge 132, a second side edge 133, and a bottom edge 134. Any of the top edge 121, the first side edge 122, the second side edge 123, and the bottom edge 124 of the anterior shell 120 may flair outwardly from the torso to change the stiffness and/or reduce chaffing on the torso. Likewise, any of the top edge 131, the first side edge 132, the second side edge 133, and the bottom edge 134 of the posterior shell 130 may flair outwardly from the torso to change the stiffness and/or reduce chaffing on the torso.

The anterior shell 120 and the posterior shell 130 are formed of a rigid material, and the stiffness of the anterior shell 120 and the posterior shell 130 may be changed by changing the type and/or thickness of materials. Preferably, each shell is formed of a plastic material such as a molded polyethylene or polypropylene. Preferably, at least five different sizes of the shells may be provided, for example, extra small, small, medium, large and extra large, or optionally, further sizes in-between may be provided, to accommodate the torsos of individuals of different sizes including variations in height and girth. Thus, multiple sizes for the anterior shell 120 and the posterior shell 130 are provided, and the sizes for the anterior shell 120 and the posterior shell 130 are interchangeable.

The anterior shell 120 and the posterior shell 130 are secured on opposite sides of the patient's torso by a fastening system 140 interconnecting the anterior shell 120 and the posterior shell 130. While many different types of fastening systems may be employed to secure the shells about the torso, Velcro™-type straps are preferred. One end of each strap is secured to one of the shells. The straps extend to mating portions of the Velcro™ fastener secured on the opposite shell. Alternative fastening systems include strap/buckle combinations and elastic materials.

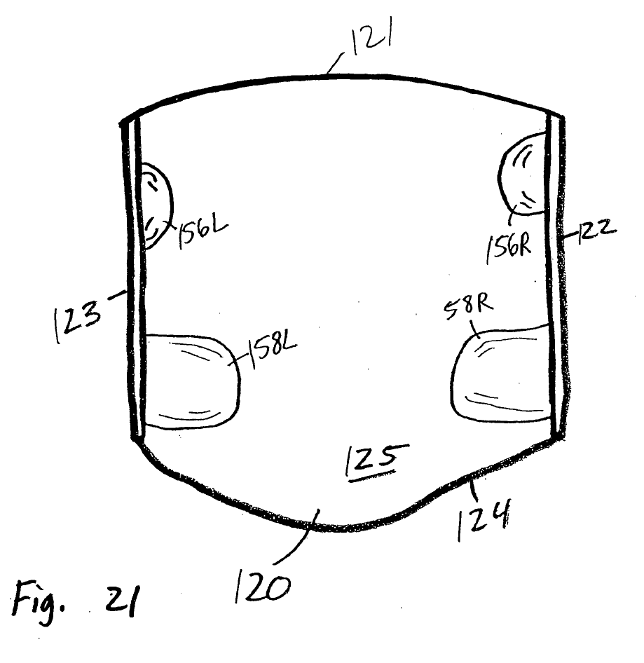

Turning now to FIG. 21, there is shown a view of the interior surface 125 of the anterior shell 120. Attached to the interior surface 125 of the anterior shell 120 are a number of anterior pressure pads. A pair of rib pads 156L and 156R are attached to the interior surface 125 of the anterior shell 120 at opposite sides of the top portion of the interior surface 125. A pair of anterior superior iliac spine (ASIS) pads 158L and 158R are attached to the interior surface 125 of the anterior shell 120 at opposite sides of the bottom portion of the interior surface 125. Each of the anterior pressure pads, which are the pair of rib pads 56L,56R, and the pair of anterior superior iliac spine (ASIS) pads 58L,58R in the embodiment of the lumbosacral orthosis 110 shown, extend away from the interior surface 125 of the anterior shell 120.

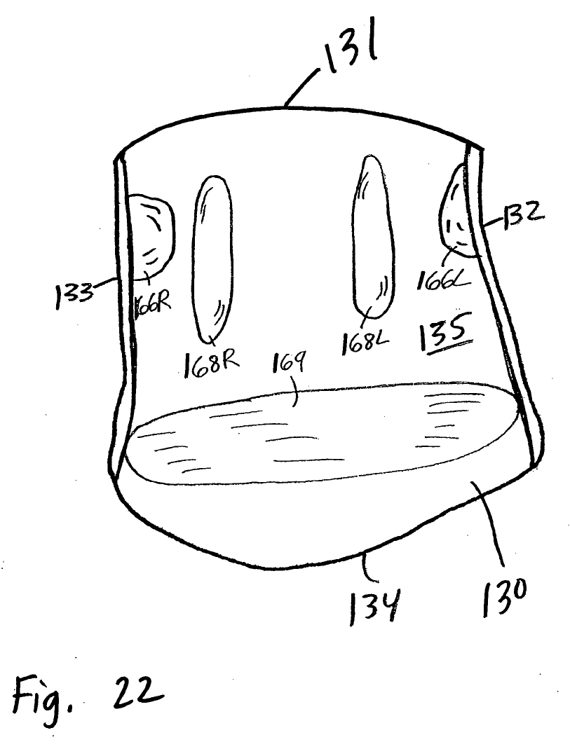

Turning now to FIG. 22, there is shown a view of the interior surface 135 of the posterior shell 130. Attached to the interior surface 135 of the posterior shell 130 are a number of posterior pressure pads. A pair of rib pads 166L and 166R are attached to the interior surface 135 of the posterior shell 130 at opposite sides of an upper portion of the interior surface 135. A pair of paraspinal pads 168L and 168R are attached to the interior surface 135 of the posterior shell 130 at opposed positions at the interior of the upper middle portion of the interior surface 135. A lumbar pad 169 is attached to the interior surface 135 of the posterior shell 130 at the bottom portion of the interior surface 135. Each of the posterior pressure pads, which are the pair of rib pads 66L,66R, the pair of paraspinal pads 68L, 68R, and the lumbar pad 69 in the embodiment of the lumbosacral orthosis 110 shown, extend away from the interior surface 135 of the posterior shell 130.

In the preferred embodiment of the lumbosacral orthosis 110, the anterior pressure pads each comprise an inflatable air bladder that is attached to the interior surface 125 of the anterior shell 120. Likewise, the posterior pressure pads each comprise an inflatable air bladder that is attached to the interior surface 135 of the posterior shell 130. While the anterior pressure pads and the posterior pressure pads each preferably comprise an inflatable air bladder, the invention is not limited to this type of anterior pressure pad and posterior pressure pad. While in the preferred embodiment, the anterior pressure pads and the posterior pressure pads are inflatable air bladders, the anterior pressure pads and the posterior pressure pads may also comprise liquid-filled sacs, gel-filled sacs or foam-type (e.g., polyurethane) pads.

Various means for attaching the anterior pressure pads to the interior surface 125 of the anterior shell 120 and for attaching the posterior pressure pads to the interior surface 135 of the posterior shell 130 can be provided. In one example, the means for attaching the anterior pressure pads to the interior surface 125 of the anterior shell 120 comprise (i) male Velcro™-type straps that are attached to or in-molded into the interior surface 125 of the anterior shell 120 at the desired locations for the anterior pressure pads and (ii) female Velcro™-type straps that are attached to each anterior pressure pad such that the anterior pressure pads may be attached to the interior surface 125 of the anterior shell 120 at the desired location by way of the typical Velcro™-type fastening action. In this example configuration, each anterior pressure pad can be readily attached or removed from the interior surface 125 of the anterior shell 120 depending on the individual patient's needs. Therefore, each of the anterior pressure pads shown in FIG. 21 may not be used in a treatment program. It can be appreciated that the same means for attaching the anterior pressure pads to the interior surface 125 of the anterior shell 120 would be advantageous for attaching the posterior pressure pads to the interior surface 135 of the posterior shell 130. Thus, each of the posterior pressure pads shown in FIG. 22 may not be used in a treatment program.

The orthoses according to the invention have many advantages. For example, the inflated air bladders minimize body contact and create natural air flow paths between a patient's torso and the anterior and posterior shells. As a result, the orthoses according to the invention have a more comfortable “airy” feel compared to known orthoses. The use of inflatable air bladders also allows for control over pressure on the patient's torso, mandible bones and occipital bones. For instance, an air bladder may be inflated to a smaller size for a lower pressure on the patient's body or may be inflated to a larger size for a greater pressure on the patient's body. Also, because any of the air bladders may be selected for inflation (or removed completely), the same orthosis may be used to treat different conditions (e.g., back pain or scoliosis) which require different pressure points on the patient's torso. Furthermore, the orthoses are superior to current devices because the rigid shell can be mass produced at a low expense while the air bladders provide the customizing effect which will yield the required stability.

Although the present invention has been described in considerable detail with reference to certain embodiments, one skilled in the art will appreciate that the present invention can be practiced by other than the described embodiments, which have been presented for purposes of illustration and not of limitation. For example, the described orthosis is suitable for use as a lumbosacral orthosis. Therefore, the scope of the appended claims should not be limited to the description of the embodiments contained herein.

### INDUSTRIAL APPLICABILITY

The invention relates to spinal orthoses that may be used for, among other things, immobilization following spinal surgery, correction of spinal deformities, and alleviation of low back pain.

What is claimed is:

1. A spinal orthosis comprising:
an anterior shell dimensioned for covering an anterior portion of a patient's torso such that the anterior shell extends from approximately the patient's pelvis to approximately the patient's sternum;
at least one anterior shell pressure pad extending away from an interior surface of the anterior shell;
a posterior shell dimensioned for covering a posterior portion of the patient's torso such that the posterior shell extends from approximately the patient's pelvis and terminates inferior to approximately the patient's scapular spine;
at least one posterior shell pressure pad extending away from an interior surface of the posterior shell; and
a fastening system for securing the anterior shell in covering relationship to the anterior portion of the patient's torso and for securing the posterior shell in covering relationship to the posterior portion of the patient's torso,
wherein each anterior shell pressure pad is positioned to apply pressure to a location on the patient's anterior torso and each posterior shell pressure pad is positioned to apply pressure to a location on the patient's posterior torso when the anterior shell and the posterior shell are secured to the patient's torso.

2. The spinal orthosis of claim 1 wherein:
at least one anterior shell pressure pad is an inflatable air bladder, and
at least one posterior shell pressure pad is an inflatable air bladder.

3. The spinal orthosis of claim 1 wherein:
an anterior shell pressure pad and/or a posterior shell pressure pad is positioned to apply pressure to a location on or adjacent approximately the iliac crest on a first side of the patient's torso,
an anterior shell pressure pad and/or a posterior shell pressure pad is positioned to apply pressure to a location on or adjacent approximately the axilla on the first side of the patient's torso, and
an anterior shell pressure pad and/or a posterior shell pressure pad is positioned to apply pressure to a location on or adjacent approximately to the contralateral apex on an opposite side of the patient's torso.

4. The spinal orthosis of claim 1 wherein:
an anterior shell pressure pad is positioned to apply pressure to a location on or adjacent approximately the sternum of the patient,
an anterior shell pressure pad is positioned to apply pressure to a location on or approximately adjacent the left anterior superior iliac spine of the patient,
an anterior shell pressure pad is positioned to apply pressure to a location on or adjacent approximately the right anterior superior iliac spine of the patient, and
a posterior shell pressure pad is positioned to apply pressure to approximately an interscapular location of the patient and/or a posterior shell pressure pad is positioned to apply pressure to approximately a lumbar location of the patient.

5. The spinal orthosis of claim 1 wherein:
an anterior shell pressure pad is positioned to apply pressure to a location on or adjacent approximately the sternum of the patient,
an anterior shell pressure pad is positioned to apply pressure to a location on or adjacent approximately the left anterior superior iliac spine of the patient,
an anterior shell pressure pad is positioned to apply pressure to a location on or adjacent approximately the right anterior superior iliac spine of the patient, and
a posterior shell pressure pad is positioned to apply pressure to approximately a paraspinal location of the patient.

6. The spinal orthosis of claim 1 wherein:
at least one of the anterior pressure pads is removably secured to the interior surface of the anterior shell, and/or at least one of the posterior pressure pads is removably secured to the interior surface of the posterior shell.

7. The spinal orthosis of claim 1 wherein:
at least one anterior shell pressure pad is a liquid-filled sac or a gel-filled sac, and/or
at least one posterior shell pressure pad is a liquid-filled sac or a gel-filled sac.

8. The spinal orthosis of claim 1 wherein:
at least one anterior shell pressure pad is a foam pad, and/or
at least one posterior shell pressure pad is a foam pad.

9. The spinal orthosis of claim 1 wherein the orthosis comprises:
a sternal anterior shell pressure pad secured to an upper intermediate section of the interior surface of the anterior shell,
a first anterior superior iliac spine anterior shell pressure pad secured to a lower first lateral section of the interior surface of the anterior shell,
a second anterior superior iliac spine anterior shell pressure pad secured to a lower second lateral section of the interior surface of the anterior shell, the lower second lateral section being opposite the lower first lateral section, and
a scapular posterior shell pressure pad secured to an upper intermediate section of the interior surface of the posterior shell.

10. The spinal orthosis of claim 9 wherein the orthosis further comprises:
a pair of spaced apart paraspinal posterior shell pressure pads secured to a central intermediate section of the interior surface of the posterior shell below the scapular posterior shell pressure pad.

11. The spinal orthosis of claim 10 wherein:
the paraspinal posterior shell pressure pads are oblong and have a longitudinal axis extending toward a top edge of the posterior shell.

12. The spinal orthosis of claim 9 wherein:
the scapular posterior shell pressure pad is T-shaped.

13. The spinal orthosis of claim 9 wherein the orthosis further comprises:
a lumbar posterior shell pressure pad secured to a lower intermediate section of the interior surface of the posterior shell.

14. The spinal orthosis of claim 1 wherein the orthosis comprises:
a sternal anterior shell pressure pad secured to an upper intermediate section of the interior surface of the anterior shell,
a first anterior superior iliac spine anterior shell pressure pad secured to a lower first lateral section of the interior surface of the anterior shell,
a second anterior superior iliac spine anterior shell pressure pad secured to a lower second lateral section of the interior surface of the anterior shell, the lower second lateral section being opposite the lower first lateral section, and
a lumbar posterior shell pressure pad secured to a lower intermediate section of the interior surface of the posterior shell.

15. The spinal orthosis of claim 14 wherein:
the sternal anterior shell pressure pad is T-shaped.

16. The spinal orthosis of claim 1 wherein the orthosis comprises:
a rib anterior shell pressure pad secured to an intermediate lateral section of the interior surface of the anterior shell,
a rib posterior shell pressure pad secured to an intermediate lateral section of the interior surface of the posterior shell,
an axillary anterior shell pressure pad secured to an upper lateral section of the interior surface of the anterior shell,
an axillary posterior shell pressure pad secured to an upper lateral section of the interior surface of the posterior shell, and
a lumbar posterior shell pressure pad secured to a lower intermediate section of the interior surface of the posterior shell.

17. The spinal orthosis of claim 1 wherein the orthosis comprises:
a pair of axillary anterior shell pressure pads secured to opposed upper lateral sections of the interior surface of the anterior shell,
a sternal anterior shell pressure pad secured to an upper intermediate section of the interior surface of the anterior shell,
a pair of rib anterior shell pressure pads secured to opposed intermediate lateral sections of the interior surface of the anterior shell,
a pair of anterior superior iliac spine anterior shell pressure pads secured to opposed lower second lateral sections of the interior surface of the anterior shell,
a pair of axillary posterior shell pressure pads secured to opposed upper lateral sections of the interior surface of the posterior shell,
a scapular posterior shell pressure pad secured to an upper intermediate section of the interior surface of the posterior shell,
a pair of rib posterior shell pressure pads secured to opposed intermediate lateral sections of the interior surface of the posterior shell,
a lumbar posterior shell pressure pad secured to a lower intermediate section of the interior surface of the posterior shell, and
a pair of spaced apart paraspinal posterior shell pressure pads secured to a central intermediate section of the interior surface of the posterior shell below the scapular posterior shell pressure pad.

18. The spinal orthosis of claim 1 wherein the orthosis further comprises:
a mandibular support secured to and extending upward from the anterior shell,
at least one mandibular support pressure pad extending away from an interior surface of the mandibular support,
an occipital support secured to and extending upward from the posterior shell, and
at least one occipital support pressure pad extending away from an interior surface of the occipital support.

19. The spinal orthosis of claim 18 wherein the orthosis comprises:
a pair of spaced apart mandibular support pressure pads extending away from the interior surface of the mandibular support, and
a pair of spaced apart occipital support pressure pads extending away from the interior surface of the occipital support.

20. The spinal orthosis of claim 1 wherein the orthosis further comprises:
an anterior thigh support,
a posterior thigh support secured to and extending downward from the posterior shell, and
a fastening system for securing the anterior thigh support in covering relationship to an anterior portion of the patient's thigh and for securing the posterior thigh support in covering relationship to a posterior portion of the patient's thigh.

21. The spinal orthosis of claim 20 wherein:
the posterior thigh support is secured to the posterior shell with a hinge.

22. The spinal orthosis of claim 21 wherein the orthosis further comprises:
at least one anterior thigh support pressure pad extending away from an interior surface of the anterior thigh support, and
at least one posterior thigh support pressure pad extending away from an interior surface of the posterior thigh support.

23. The spinal orthosis of claim 1 wherein the spinal orthosis is a thoracolumbosacral orthosis.

24. The spinal orthosis of claim 1 wherein the spinal orthosis is a lumbosacral orthosis.

25. The spinal orthosis of claim 1 wherein the spinal orthosis is a cervical-thoracic-lumbar-sacral orthosis.

26. A cervical collar orthosis comprising:
an anterior shell having a mandibular support region dimensioned for contacting a patient's mandible and having a clavicular support region dimensioned for contacting the patient's collar bones;
at least one anterior shell pressure pad extending away from an interior surface of the anterior shell;
a posterior shell having an occipital support region dimensioned for contacting the patient's occipital bones and having a lower posterior support region dimensioned for contacting the patient's scapular spine and/or neck;
at least one posterior shell pressure pad extending away from an interior surface of the posterior shell; and
a fastening system for securing the anterior shell in covering relationship to an anterior portion of the patient's neck region and for securing the posterior shell in covering relationship to a posterior portion of the patient's neck region,
wherein at least one anterior shell pressure pad is an inflatable air bladder, and at least one posterior shell pressure pad is an inflatable air bladder.

27. The cervical collar orthosis of claim 26 wherein the orthosis comprises:
at least one mandibular support pressure pad extending away from the interior surface of the anterior shell at the mandibular support region of the anterior shell.

28. The cervical collar orthosis of claim 26 wherein the orthosis comprises:
a pair of laterally spaced mandibular support pressure pads extending away from the interior surface of the anterior shell at the mandibular support region of the anterior shell.

29. The cervical collar orthosis of claim 26 wherein the orthosis comprises:
at least one clavicular support pressure pad extending away from the interior surface of the anterior shell at the clavicular support region of the anterior shell.

30. The cervical collar orthosis of claim 26 wherein the orthosis comprises:
a pair of laterally spaced clavicular support pressure pads extending away from the interior surface of the anterior shell at the clavicular support region of the anterior shell.

31. The cervical collar orthosis of claim 26 wherein the orthosis comprises:
at least one occipital support pressure pad extending away from the interior surface of the posterior shell at the occipital support region of the posterior shell.

32. The cervical collar orthosis of claim 26 wherein the orthosis comprises:
a pair of laterally spaced occipital support pressure pads extending away from the interior surface of the posterior shell at the occipital support region of the posterior shell.

33. The cervical collar orthosis of claim 26 wherein the orthosis comprises:
at least one lower posterior support pressure pad extending away from the interior surface of the posterior shell at the lower posterior support region of the posterior shell.

34. The cervical collar orthosis of claim 26 wherein the orthosis comprises:
a pair of laterally spaced lower posterior support pressure pads extending away from the interior surface of the posterior shell at the lower posterior support region of the posterior shell.
